(19) United States
(12) Patent Application Publication
(10) Pub. No.: US 2009/0125096 A1
(43) Pub. Date: May 14, 2009

(54) Stent Graft With Pins

(75) Inventors: Jack Chu, Santa Rosa, CA (US); Brendan Cunniffe, Oranmore (IE); Matthew Rust, Santa Rosa, CA (US); Trevor Greenan, Santa Rosa, CA (US); Frank Yang, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/938,642

(22) Filed: Nov. 12, 2007

(51) Int. Cl. A61F 2/06

(52) U.S. Cl. 623/1.14

(57) ABSTRACT

A stent graft with pins, a stent graft including a tubular graft having a perimeter and a central axis; at least one stent ring operably connected about the perimeter, the stent ring having a plurality of struts connected in a sinusoidal pattern, at least one of the plurality of struts having a hole; and a pin having a free end. The pin is secured in the hole with the free end directed outwardly from the central axis.

### TECHNICAL FIELD

The technical field of this disclosure is medical implantation devices, particularly, a stent graft with pins.

### BACKGROUND OF THE INVENTION

Stent grafts have been developed for the treatment of abdominal aortic aneurysms. An abdominal aortic aneurysm is a bulge that forms in the wall of the abdominal aorta, which is the main vessel of the arterial system of the body that extends through the abdomen. Abdominal aortic aneurysms can lose elasticity over time and rupture under normal blood pressure. A stent graft is a woven tube (graft) supported by a tubular metal stent. The stent graft is placed inside of an aneurysmal vessel to exclude the abdominal aortic aneurysm from normal blood flow and reduces pressure on the aneurysmal vessel. Stent grafts employ sealing regions at the proximal and distal ends to seal the stent graft to the normal aortic wall and prevent blood flow between the stent graft and the aneurysmal vessel. The sealing regions can include hooks to avoid migration of the stent graft from the installed location.

One stent design uses laser cutting of Nitinol stock to form the stent with integral hooks. The hooks are bent to project outwardly from the stent before the stent graft is installed. The hooks secure the stent graft to the vessel wall. Unfortunately, blood flow applies repeated stress to the hooks, which can bend and allow the stent graft to migrate from its installed location in the abdominal aortic aneurysm.

It would be desirable to have a stent graft with pins that would overcome the above disadvantages.

### SUMMARY OF THE INVENTION

One aspect according to the present invention provides a stent graft including a tubular graft having a perimeter and a central axis; at least one stent ring operably connected about the perimeter, the stent ring having a plurality of struts connected in a sinusoidal pattern, at least one of the plurality of struts having a hole; and a pin having a free end. The pin is secured in the hole with the free end directed outwardly from the central axis.

Another aspect according to the present invention provides a stent ring including a stent ring having a central axis and a plurality of struts connected in a sinusoidal pattern, at least one of the plurality of struts having a hole; and a pin having a free end. The pin is secured in the hole with the free end directed outwardly from the central axis.

Another aspect according to the present invention provides a stent ring system including a stent ring having a central axis and a plurality of struts connected in a sinusoidal pattern, at least one of the plurality of struts having a hole; means for piercing a vessel wall, the piercing means having a free end; and means for securing the piercing means in the hole with the free end directed outwardly from the central axis.

Another aspect according to the present invention provides a method of stent ring fabrication including providing a stent ring having a central axis and a plurality of struts connected in a sinusoidal pattern, at least one of the plurality of struts having a hole; providing a pin having a free end; inserting the pin in the hole with the free end directed outwardly from the central axis; and securing the pin in the hole.

The foregoing and other features and advantages will become further apparent from the following detailed description of the presently preferred embodiments, read in conjunction with the accompanying drawings

### BRIEF DESCRIPTION OF THE DRAWINGS

FIG. 1 is a side view of a stent graft with a stent ring made in accordance with the present invention;

FIGS. 2A-2B are a side view and top view, respectively, of a stent ring made in accordance with the present invention;

FIG. 3 is a detailed view of another stent ring made in accordance with the present invention;

FIG. 4 is a detailed view of struts of a stent ring made in accordance with the present invention;

FIGS. 5A-5F are cross sectional views of pins in a stent ring made in accordance with the present invention;

FIG. 6 is a detailed view of various pin nodule embodiments for a stent ring made in accordance with the present invention; and

FIG. 7 is a detailed view of staggered pin nodules for a stent ring made in accordance with the present invention.

### DETAILED DESCRIPTION

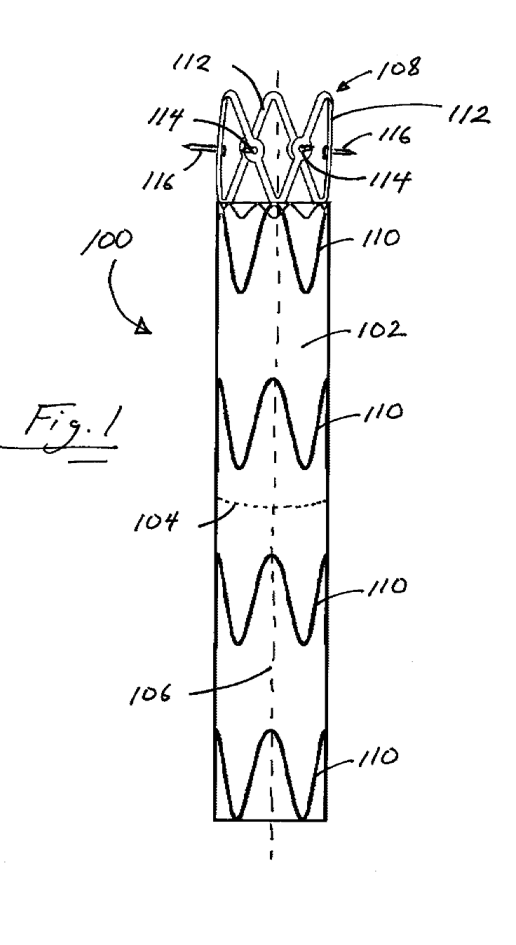

FIG. 1 is a side view of a stent graft with a stent ring made in accordance with the present invention. The stent graft 100 includes a tubular graft 102 and stent rings operably connected about the tubular graft 102. The tubular graft 102 has a perimeter 104 about a central axis 106 as indicated by the dashed lines. The central axis of the stent rings is coincident with the central axis 106. In this example, the stent graft 100 has a proximal stent ring 108 and body stent rings 110 operably connected about the perimeter 104 of the tubular graft 102. Proximal and distal are defined relative to the fluid flow in the lumen in which the stent graft is installed, with the flow being from proximal to distal. Those skilled in the art will appreciate that the stent graft can be any stent graft having stent rings. The number and axial distance between stent rings can be selected for a particular application. In one embodiment, the stent graft is a branching stent graft having a body and branches. When the stent graft includes branches, each branch has its own central axis and perimeter. In another embodiment, the stent graft includes a distal stent ring. In yet another embodiment, the stent graft includes a number of stent rings joined into a unitized stent ring.

The proximal stent ring 108 of this example has a number of struts 112 connected in a generally sinusoidal pattern. One or more of the struts 112 has a hole 114 in which a pin 116 is secured. The pins 116 pierce the vessel wall when the stent graft 100 is deployed to help secure the position of the stent graft 100 and prevent axial movement of the stent graft 100 in the vessel. In the sealing region, the pins 116 assist in maintaining the seal. Stent rings with pins can be used as any stent ring, including proximal crown rings, distal crown rings, stent rings about the stent graft body, or stent rings about stent graft branches.

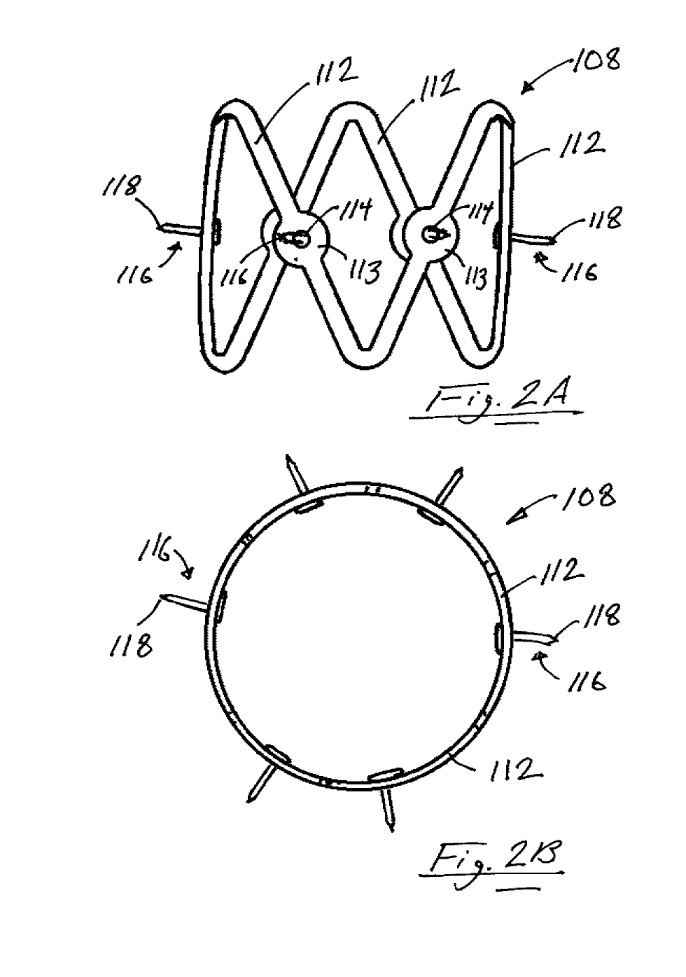

FIGS. 2A-2B, in which like elements share like reference numbers with FIG. 1, are a side view and top view, respectively, of a stent ring made in accordance with the present invention. The stent ring 108 has a number of struts 112 connected in a generally sinusoidal pattern to form a ring. In this example, each of the struts 112 includes a pin nodule 113 with a hole 114. The pins 116 are secured in the holes 114. The free end 118 of each of the pins 116 is directed outwardly from the stent ring 108 to engage the vessel when the stent graft is deployed.

FIG. 3, in which like elements share like reference numbers with FIGS. 2A-2B, is a detailed view of another stent ring made in accordance with the present invention. In this example, the stent ring 108 is a unitized stent ring in which the struts 112 form a continuous diamond pattern. The pin nodules 113 with holes 114 can be formed in the struts 112 in any regular or irregular pattern desired for a particular application.

FIG. 4, in which like elements share like reference numbers with FIGS. 2A-2B, is a detailed view of struts of a stent ring made in accordance with the present invention. In this example, the struts 112 are connected in a generally sinusoidal pattern. Each of the struts 112 includes a pin nodule 113 with a hole 114. In one embodiment, the pin nodule 113 can be the same size as the rest of the strut 112 so that the strut 112 maintains a uniform cross section, so that the holes 114 are formed in the uniform cross section. In one embodiment, the axis of the hole 114 is perpendicular to the surface of the strut 112. In another embodiment, the axis of the hole 114 is at an oblique angle to the surface of the strut 112, so that the pin can be angled relative to the surface of the strut 112.

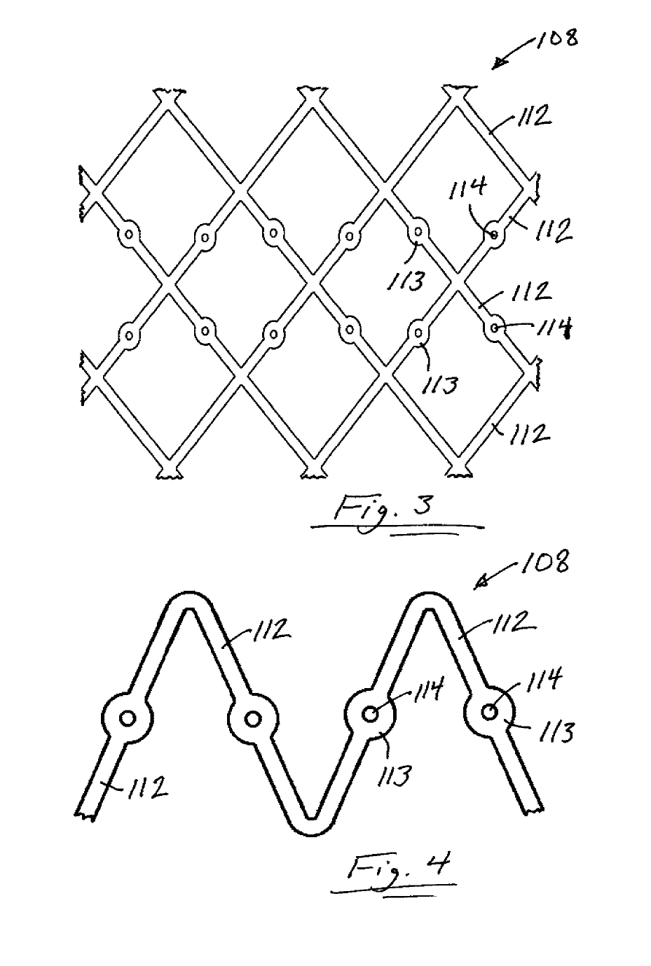

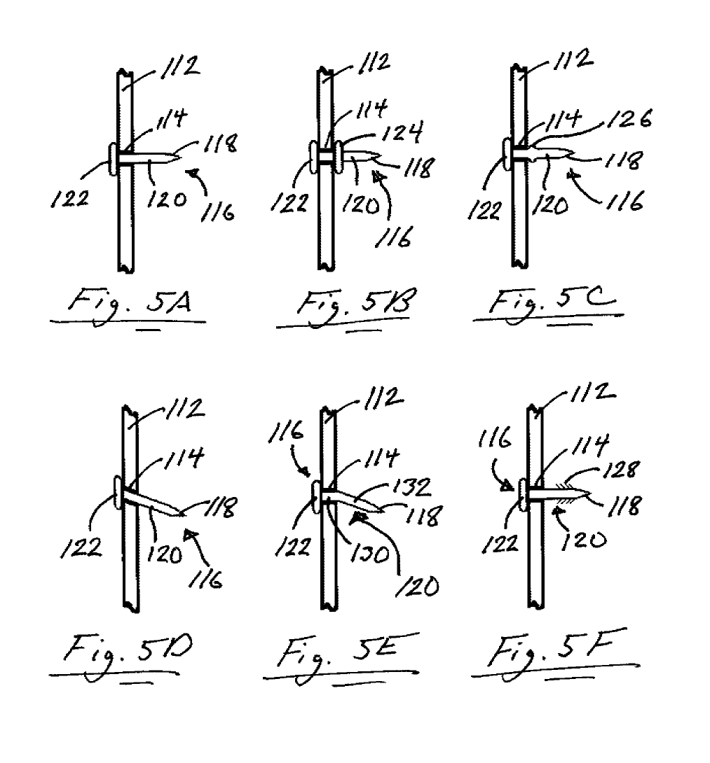

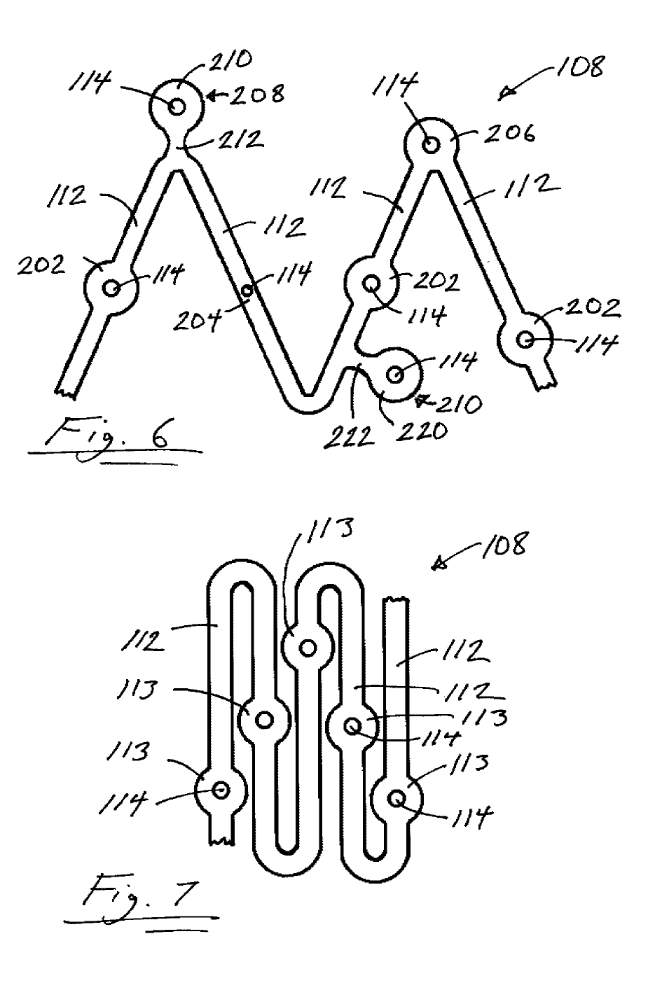

FIGS. 5A-5F, in which like elements share like reference numbers with each other and with FIGS. 2A-2B, are cross sectional views of pins in a stent ring made in accordance with the present invention. The pin 116 passes through a hole 114 in the strut 112 and is secured in place. The pin 116 includes a free end 118, a shaft 120, and a head 122. In one embodiment, the free end 118 of the pin 116 is a point. The free end 118 of the pin 116 can be sharpened into a point before or after the pin 116 is secured in the hole 114. In another embodiment, the free end 118 of the pin 116 is unsharpened so that the free end 118 has the same radial cross section as the shaft 120. The diameter of the pin 116 can be selected so that the unsharpened pin will pierce the vessel wall. Those skilled in the art will appreciate that the head 122 can have a selected profile or can be recessed in the strut 112 as desired for a particular application.

Referring to FIG. 5A, the pin 116 is secured in the hole 114. The pin 116 can be secured in the hole 114 by any method desired, such as soldering, brazing, welding, laser welding, adhesive fixing, snap fitting, friction fitting, or the like. Snap fitting uses the shape of the shaft 120 secure the pin 116. Friction fitting depends on the interference between the shaft 120 and the hole 114 to secure the pin 116. Any method keeping the pin 116 in the hole 114 and using biologically compatible materials can be used.

Referring to FIG. 5B, the pin 116 is secured in the hole 114 by a keeper 124. The keeper 124 is attached to the pin 116 by any method desired, such as soldering, brazing, welding, laser welding, adhesive fixing, snap fitting, friction fitting, or the like. The pin 116 can be rigidly fixed in the hole 114 or can be allowed some play. In one embodiment, the pin 116 is secured in the hole 114 by the keeper 124 alone. In another embodiment, the pin 116 is secured in the hole 114 by the keeper 124 in combination with fastening the pin 116 to the strut 112.

Referring to FIG. 5C, the pin 116 is secured in the hole 114 by a protrusion 126 on the shaft 120. The protrusion 126 has a large enough diameter to secure the pin 116 in the hole 114, yet is small enough to allow the protrusion 126 to pass through the hole 114 during fabrication. In one embodiment, the protrusion 126 is compressed when passing through the hole 114 and expands in the final position outside the hole 114. The pin 116 can be rigidly fixed in the hole 114 or can be allowed some play. In one embodiment, the pin 116 is secured in the hole 114 by the protrusion 126 alone. In another embodiment, the pin 116 is secured in the hole 114 by the protrusion 126 in combination with fastening the pin 116 to the strut 112.

Referring to FIG. 5D, the pin 116 is at an oblique angle to the strut 112. In this embodiment, the axis of the hole 114 is at an oblique angle to the surface of the strut 112, so that the pin 116 is angled relative to the surface of the strut 112. The orientation of the free end 118 relative to the central axis of the stent graft can be selected to preferentially resist forces from a given direction, such as the force from proximal flow. In one embodiment, the pin 116 is secured in the hole 114 by soldering, brazing, welding, laser welding, adhesive fixing, snap fitting, friction fitting, or the like. In another embodiment, the pin 116 is secured in the hole 114 with a keeper or by a protrusion on the shaft 120.

Referring to FIG. 5E, the shaft 120 of the pin 116 includes a straight portion 130 and an angled portion 132. The angled portion 132 can be formed by bending the shaft of a straight pin such as illustrated in FIG. 5A. Referring to FIG. 5E, the orientation of the free end 118 relative to the central axis of the stent graft can be selected to preferentially resist forces from a given direction, such as the force from proximal flow. In one embodiment, the pin 116 is secured in the hole 114 by interference between the angled portion 132 and the strut 112. In another embodiment, the pin 116 is secured in the hole 114 is secured in the hole 114 by soldering, brazing, welding, laser welding, adhesive fixing, snap fitting, friction fitting, or the like. In another embodiment, the pin 116 is secured in the hole 114 with a keeper or by a protrusion on the shaft 120.

Referring to FIG. 5F, the pin 116 includes barbs 128. The barbs 128 increase the holding power of the pin 116 in the vessel wall when the stent graft is deployed in the vessel.

The strut 112 of the stent ring and the pin 116 can be made of any biocompatible materials suitable for a particular application. In one embodiment, the strut 112 and the pin 116 are made of the same material. In another embodiment, the strut 112 and the pin 116 are made of different materials. Exemplary materials for the strut 112 and/or the pin 116 include stainless steels, such as 316L stainless steel; shape memory alloys, such as nitinol; and nickel-cobalt-chromium-molybdenum alloy, such as MP35N® alloy available from SPS Technologies, Inc., of Jenkintown, Pa.; or the like. Those skilled in the art will appreciate that many materials and combinations of materials can be used as desired for a particular application.

FIG. 6, in which like elements share like reference numbers with FIGS. 2A-2B, is a detailed view of various pin nodule embodiments for a stent ring made in accordance with the present invention. The various pin nodule embodiments are illustrated on a portion of a single stent ring 108 for ease of illustration. A single type of pin nodule can be used on one stent ring or a mixture of pin nodule types can be used on one stent ring. The pin nodule as defined herein is any portion of the stent ring having a hole in which a pin can be secured.

Exemplary types of pin nodules discussed below are widened pin nodules 202, uniform pin nodules 204, crown stent modules 206, crown extended pin nodules 208, and strut extended pin nodules 210.

In one embodiment, the pin nodule is in line with the strut 112. Widened pin nodule 202 is a widened portion of the strut 112. The widening strengthens the strut 112 to allow for the material removed in forming the hole 114. The widened pin nodule 202 can be located anywhere along the length of the strut 112. Uniform pin nodule 204 is a normal width portion of the strut 112 having a hole 114. The uniform pin nodule 204 can be located anywhere along the length of the strut 112. Crown stent module 206 is a widened portion of the stent ring 108 at the crown of the stent ring where one strut 112 meets an adjacent strut 112.

In another embodiment, the pin nodule extends the strut 112. Crown extended pin nodule 208 includes a pin nodule body 210 having a hole 114, and an pin nodule extension 212 joining the pin nodule body 210 to the crown of the stent ring 108 where one strut 112 meets an adjacent strut 112. Strut extended pin nodule 218 includes a pin nodule body 220 having a hole 114, and an pin nodule extension 222 joining the pin nodule body 220 to a strut 112 of the stent ring 108.

Those skilled in the art will appreciate that the placement and shape of the pin nodules on the stent ring 108 depends on a number of factors, such as the radial force required when the stent graft is installed, localized strain on the stent ring 108 when the stent ring 108 is compressed and expanded during manufacture and deployment, localized strain on the stent ring 108 when the stent graft is in use, and the like. In one example, a widened pin nodule 202 located mid-length on the strut 112 is good for applying radial force to keep the pin anchored in the vessel and located in a low strain region. In another example, the crown extended pin nodule 208 is good because by being positioned at the extreme end of the device it is more likely to engage healthy tissue, but is less able to apply radial force to keep the pin anchored in the vessel because it is on the tip of the stent ring 108. In yet another example, the crown stent module 206 is located in a high strain region, so the design and material selection must account for the high strain when designing the crown stent module.

FIG. 7, in which like elements share like reference numbers with FIGS. 2A-2B, is a detailed view of staggered pin nodules for a stent ring made in accordance with the present invention. In this example, the stent ring 108 is in the compressed condition before deployment. The pin nodules 113 are staggered along the length of the struts 112 so that the pin nodules 113 on adjacent struts 112 do not interfere with each other. The staggered pin nodules allow the stent ring 108 to be compressed to a smaller diameter than would be possible then if the pin nodules 113 on adjacent struts 112 were at the same position on the struts 112 and made contact with each other when the stent ring 108 is compressed.

The stent ring and stent graft can be manufactured using the techniques applied to conventional as suitable to the materials selected. One method includes providing a stent ring having a central axis and struts connected in a sinusoidal pattern, with one or more of the struts having a hole through the strut; providing a pin having a free end; inserting the pin in the hole with the free end directed outwardly from the central axis; and securing the pin in the hole. In one embodiment, the stent ring is formed from tube stock, such as laser cutting from tube stock. In another embodiment, the stent ring is formed from flat stock and fashioned into a ring, such as laser cutting from flat stock and forming into a stent ring. The axis of hole can be perpendicular or at an oblique angle to the surface of the strut. The pin can be secured in the hole by a method of connecting the parts, such as soldering, brazing, welding, laser welding, or adhesive fixing, or a method relying on the dimensions of the parts, such as snap fitting or friction fitting. In one embodiment, the free end of the pin is sharpened into a point before the pin is secured in the hole. In another embodiment, the free end of the pin is sharpened into a point after the pin is secured in the hole. In yet another embodiment, the free end of the pin is unsharpened so that the free end has the same radial cross section as the shaft. In one embodiment, the shaft of the pin can be bent after the pin is inserted in the hole so that the free end of the pin is at an oblique angle to the surface of the stent. The stent rings can be fastened to the graft material with thread, adhesive, or the like to form the stent graft. Those skilled in the art will appreciate that the fabrication can be tailored to the particular materials used.

The stent graft is delivered to the aneurysm in a compressed condition with the sharp free ends of the pins protected from the vessel wall by a catheter capsule or a specialty shaped sheath which avoids engaging contact with the sharp ends of the pins. Once released from its compressed condition the stent graft is allowed to expand or expanded so that the pins pierce the vessel wall. For the example of an abdominal aortic aneurysm, a catheter is advanced to the abdominal aortic aneurysm through the femoral artery, the carotid artery, or the subclavian artery. The catheter is guided to the location of the aneurysm with X-ray or fluoroscopic data and the stent graft advanced to the aneurysm through the catheter. When stent graft is outside the catheter and in the aneurysm, the stent graft can be allowed to expand or expanded. In one embodiment, the stent rings of the stent graft are made of a shape memory alloy, such as nitinol, that expands the stent graft to a predetermined shape when the stent rings are exposed to body temperature. In another embodiment, the stent rings of the stent graft are made of elastic alloy and held compressed with dissolvable ties. The dissolvable ties dissolve and the stent graft expands when the dissolvable ties are exposed to the fluid in the vessel. In another embodiment, the stent rings of the stent graft are made of deformable alloy and expanded with a balloon, such as a balloon used in percutaneous transluminal coronary angioplasty (PTCA). The pins in the stent graft ring pierce the vessel wall to secure and/or seal the stent graft in the aneurysm. Those skilled in the art will appreciate that the stent graft can be used with any aneurysm in the body and is not limited to use with abdominal aortic aneurysms.

While specific embodiments of the invention are disclosed herein, various changes and modifications can be made without departing from the spirit and scope of the invention. The scope of the invention is indicated in the appended claims, and all changes that come within the meaning and range of equivalents are intended to be embraced therein.

1. A stent graft comprising:
a tubular graft having a perimeter and a central axis;
at least one stent ring operably connected about the perimeter, the stent ring having a plurality of struts connected in a sinusoidal pattern, at least one of the plurality of struts having a hole; and
a pin having a free end;
wherein the pin is secured in the hole with the free end directed outwardly from the central axis.

2. The stent graft of claim 1 further comprising a plurality of stent rings connected in a diamond pattern.

3. The stent graft of claim 1 wherein the free end is a point.

4. The stent graft of claim 1 wherein the pin is secured in the hole with a method selected from the group consisting of soldering, brazing, welding, laser welding, and adhesive fixing.

5. The stent graft of claim 1 wherein the pin is secured in the hole with a method selected from the group consisting of snap fitting and friction fitting.

6. The stent graft of claim 1 wherein the pin has a head opposite the free end and a shaft between the head and the free end, and further comprising a keeper about the shaft securing the pin in the hole.

7. The stent graft of claim 1 wherein the pin has a head opposite the free end and a shaft between the head and the free end, and further comprising a protrusion about the shaft securing the pin in the hole.

8. The stent graft of claim 1 wherein the strut has a surface and the hole is oblique to the surface.

9. The stent graft of claim 1 wherein the pin has a head, a straight portion adjacent the head, and an angled portion adjacent the free end, and the straight portion is disposed in the hole.

10. The stent graft of claim 1 wherein the pin has a shaft adjacent the free end, further comprising barbs on the shaft.

11. The stent graft of claim 1 wherein at least one of the plurality of struts has a pin nodule, the hole being in the pin nodule.

12. The stent graft of claim 11 wherein the pin nodule is selected from the group consisting of widened pin nodules, uniform pin nodules, and crown stent nodules.

13. The stent graft of claim 11 wherein the pin nodule comprises a pin nodule extension and a pin nodule body, the pin nodule extension extending the at least one of the plurality of struts and being connected to the pin nodule body having the hole.

14. The stent graft of claim 1 wherein at least two adjacent struts of the plurality of struts have pin nodules, the pin nodules being axially staggered from each other along the two adjacent struts.

15. The stent graft of claim 1 wherein the pin and the at least one of the plurality of struts having the hole are made of different materials.

16. The stent graft of claim 1 wherein the pin and at least one of the plurality of struts having the hole are made of one material.

17. The stent graft of claim 1 wherein the pin is made of a material selected from the group consisting of stainless steel, shape memory alloy, and nickel-cobalt-chromium-molybdenum alloy.

18. The stent graft of claim 1 wherein the plurality of struts is made of a material selected from the group consisting of stainless steel, shape memory alloy, and nickel-cobalt-chromium-molybdenum alloy.

19. A stent ring comprising:
a stent ring having a central axis and a plurality of struts connected in a sinusoidal pattern, at least one of the plurality of struts having a hole; and
a pin having a free end;
wherein the pin is secured in the hole with the free end directed outwardly from the central axis.

20. The stent graft of claim 19 further comprising a plurality of stent rings connected in a diamond pattern.

21. The stent graft of claim 19 wherein the free end is a point.

22. The stent graft of claim 19 wherein the pin is secured in the hole with a method selected from the group consisting of soldering, brazing, welding, laser welding, and adhesive fixing.

23. The stent graft of claim 19 wherein the pin is secured in the hole with a method selected from the group consisting of snap fitting and friction fitting.

24. The stent graft of claim 19 wherein the pin has a head opposite the free end and a shaft between the head and the free end, and further comprising a keeper about the shaft securing the pin in the hole

25. The stent graft of claim 19 wherein the pin has a head opposite the free end and a shaft between the head and the free end, and further comprising a protrusion about the shaft securing the pin in the hole.

26. The stent graft of claim 19 wherein the strut has a surface and the hole is oblique to the surface.

27. The stent graft of claim 19 wherein the pin has a head, a straight portion adjacent the head, and an angled portion adjacent the free end, and the straight portion is disposed in the hole.

28. The stent graft of claim 19 wherein the pin has a shaft adjacent the free end, further comprising barbs on the shaft.

29. The stent graft of claim 19 wherein at least one of the plurality of struts has a pin nodule, the hole being in the pin nodule.

30. The stent graft of claim 29 wherein the pin nodule is selected from the group consisting of widened pin nodules, uniform pin nodules, and crown stent nodules.

31. The stent graft of claim 29 wherein the pin nodule comprises a pin nodule extension and a pin nodule body, the pin nodule extension extending the at least one of the plurality of struts and being connected to the pin nodule body having the hole.

32. The stent graft of claim 19 wherein at least two adjacent struts of the plurality of struts have pin nodules, the pin nodules being axially staggered from each other along the two adjacent struts.

33. The stent graft of claim 19 wherein the pin and at least one of the plurality of struts having the hole are made of different materials.

34. The stent graft of claim 19 wherein the pin and at least one of the plurality of struts having the hole are made of one material.

35. The stent graft of claim 19 wherein the pin is made of a material selected from the group consisting of stainless steel, shape memory alloy, and nickel-cobalt-chromium-molybdenum alloy.

36. The stent graft of claim 19 wherein the plurality of struts is made of a material selected from the group consisting of stainless steel, shape memory alloy, and nickel-cobalt-chromium-molybdenum alloy.

37. A stent ring system comprising:
a stent ring having a central axis and a plurality of struts connected in a sinusoidal pattern, at least one of the plurality of struts having a hole;
means for piercing a vessel wall, the piercing means having a free end; and
means for securing the piercing means in the hole with the free end directed outwardly from the central axis.

38. The method of claim 37 further comprising means for retaining the piercing means in the vessel wall.

39. A method of stent ring fabrication comprising:
providing a stent ring having a central axis and a plurality of struts connected in a sinusoidal pattern, at least one of the plurality of struts having a hole;
providing a pin having a free end;
inserting the pin in the hole with the free end directed outwardly from the central axis; and
securing the pin in the hole.

40. The method of claim 39 wherein the providing a stent ring comprises laser cutting the stent ring from tube stock.

41. The method of claim 39 wherein the securing comprises securing the pin in the hole by a method selected from the group consisting of soldering, brazing, welding, laser welding, and adhesive fixing.

42. The method of claim 39 wherein the securing comprises securing the pin in the hole by a method selected from the group consisting of snap fitting and friction fitting.

43. The method of claim 39 further comprising sharpening the free end.
